(19) United States
(12) Patent Application Publication
(10) Pub. No.: US 2021/0315227 A1
(43) Pub. Date: Oct. 14, 2021

(54) STABILIZED COMPOSITIONS CONTAINING EDIBLE OIL AND THEIR USES IN FOOD PRODUCTS

(71) Applicant: ROQUETTE FRERES, Lestrem (FR)

(72) Inventors: Bernard PORA, Shanghai (CN); Chaolan TANG, Wuhan (CN); Jovin HASJIM, Shanghai (CN)

(21) Appl. No.: 17/271,733

(22) PCT Filed: Sep. 3, 2019
(86) PCT No.: PCT/IB2019/000995

§ 371 Date: Feb. 26, 2021

(30) Foreign Application Priority Data
Sep. 4, 2018 (CN) 201811026421.6

(51) Int. Cl. A23D 9/06; A23D 9/007; A23D 9/05; A23L 33/12; A23L 33/125; A23L 33/19; A23L 33/15; A23L 33/16; A23L 2/66; C11B 5/00; A61K 9/00; A61K 36/02; A61K 35/60; A61K 35/612; A61K 9/16; A61K 31/202

(52) U.S. Cl. CPC A23D 9/06; A23V 2002/00; A23D 9/05; A23L 33/12; A23L 33/125; A23L 33/19; A23L 33/15; A23L 33/16; A23L 2/66; C11B 5/0021; A61K 9/0056; A61K 36/02; A61K 35/60; A61K 35/612; A61K 9/1623; A61K 9/1617; A61K 31/202; A23D 9/007

(57) ABSTRACT

The present invention relates to a composition comprising:
(i) from 10 to 40% by weight, preferably from 20 to 30% by weight of an edible oil;
(ii) lactose and
(iii) mannitol

wherein the edible oil comprises at least 20% by weight, preferably between 25 and 40% by weight, typically 30 and 35% by weight of polyunsaturated fatty acids and the weight ratio of lactose to mannitol is from 1:3 to 3:1, preferably between 1:2 to 2:1, more preferably 1:1.

The present invention relates to stabilized compositions containing edible oil, the process for obtaining such compositions, the use of such compositions and the food products containing thereof.

Edible oils that contain unsaturated fatty acids, and especially polyunsaturated fatty acids (PUFA, such as omega-3 and omage-6), usually in the form of glyceride esters, have been shown to have beneficial health effects. These health effects include reduction of cholesterol levels, protection against coronary heart disease and suppression of platelet aggregation. For example, fish oil, which contains the omega-3 and omega-6 fatty acids docosahexaenoic acid (DHA) and eicosapentaenoic acid (EPA), has been used in food products and in nutritional products for its health benefits. Microalgal oil, which is an alternative source of DHA, has also been described as having an anti-obesity effect.

However, PUFA have a tendency to undergo oxidation and as a consequence can have an unpleasant taste and/or an odour, such as fishy odour, and reduced bioavailability. Besides, the storage stability of products containing PUFA is also relatively short because of the problems associated by the tendency to undergo oxidation. Several solutions trying to stabilize PUFA against oxidation have therefore already been disclosed in the prior art. For example, U.S. Pat. No. 4,963,385 describes a stabilizer system against oxidative attack by using a sugar or a sugar alcohol, possibly in combination with a metal-ion chelator. The combination with a metal-ion chelator is described as providing a synergistic effect against the oxidation of fish oil. WO94/01001 discloses a microencapsulated oil (preferably fish oil) or fat product on the basis of caseinate as the encapsulating compound. The use of caseinate as the only emulsifying agent optionally in combination with at least one carbohydrate results in relatively stable oil or fat product. U.S. Pat. No. 4,438,106 and U.S. Pat. No. 6,638,557 suggest stabilizing fish oil by using cyclodextrin, although it can increase the viscosity of the composition, which can be a disadvantage if the composition is used for powder production. US2008/0138493 discloses the use of the combination of one or more sugar alcohols (for example mannitol) with one or more reducing sugars (for example glucose syrup) to increase the stability of edible oil, preferably fish oil. Some polyols have also been disclosed as having an antioxidant activity, for example Faraji H. and Lindsay R. disclose, in J. Agric. Food Chem, 2004, 54, 7164-7171, that antioxidant activity was confirmed for fructose, sucrose, raffinose, sorbitol or mannitol when incorporated at 16% of the aqueous phase into model fish oil-in-water emulsions. Fructose is notably mentioned as having the highest antioxidant activity and mannitol as having the lowest antioxidant capacity. It is also to be noted that fructose and mannitol are sweeteners, and their uses can affect the taste of a composition. Although the stabilization of PUFA against oxidation has been discussed in the prior art, there is still a need for a stabilized composition containing edible oil and a need for preventing the oxidation of edible oil, notably when the edible oil is microalgal oil. Such composition needs also to have a good taste (i.e the composition has an improved stability against rancidity), a good odour (particularly after a storage), a stable DHA content (particularly during a storage), while allowing it to be easily encapsulated and spray-dried into powder.

The present invention relies on the unexpected results of the inventors showing that a composition containing edible oil can be stabilized by a particular weight ratio of lactose to mannitol. These unexpected results show not only that a composition having this particular weight ratio of lactose to mannitol (i) prevent and/or protect the edible oil from oxidation, but also that (ii) this composition has a good taste, a good odour (even after a storage), a stable DHA content (particularly during a storage), while allowing it to be easily encapsulated and spray-dried into powder.

In a first aspect, the present invention relates then to a composition comprising:
(i) from 10 to 40% by weight, preferably from 20 to 30% by weight of an edible oil;
(ii) lactose and
(iii) mannitol
   wherein the edible oil comprises at least 20% by weight, preferably between 25 and 40% by weight, typically 30 and 35% by weight of polyunsaturated fatty acids, and wherein the weight ratio of lactose to mannitol is from 1:3 to 3:1, preferably between 1:2 to 2:1, more preferably 1:1.

As used herein, the term “edible oil” means oil that is non-toxic and that can be consumed. The edible oil is preferably capable of providing health benefits. The edible oil is a triglyceride composition which is typically liquid within the temperature range of 0° C. to 25° C. at an atmospheric pressure. The oil is generally hydrophobic, obtained or obtainable from a natural source, such as a vegetable oil or animal oil. The oil may be a mixture of different oils from different sources. In a preferred embodiment the edible oil is fish oil, a krill oil, a microalgal oil, and is more preferably a microalgal oil. The edible oil corresponds to 10 to 40% by weight of the composition. As used herein, “from 10 to 40%, preferably from 20 to 30%” means all the values between 10 to 40%, i.e. 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% and 40%.

In a preferred embodiment, the polyunsaturated fatty acids are chosen in the group consisting of docosahexaenoic acid (DHA), eicosapentaenoic acid (EPA), arachidonic acid (ARA), docosapentaenoic acid (DPA), stearidonic acid, linolenic acid and a mixture thereof, preferably selected from DHA, EPA and mixtures thereof, more preferably DHA. As used herein, the term “lactose” refers to a disaccharide sugar composed of galactose and glucose that is found in milk. For example, it can be precipitated from whey using ethanol, and then isolated.

As used herein, the term “mannitol” refers to a sugar alcohol, i.e. a polyol obtainable by reduction of saccharides, for example by the hydrogenation of mannose or fructose. In a preferred embodiment, the composition of the invention is spray-dried. Before spray drying, 45-55% of water (based on dry substance of the composition) is added along with other components (encapsulation agent, emulsifier, free-flowing agent and chelating agent) to make an emulsion. The residual moisture in the obtained powder after spray drying is 2-3% of moisture. The water activity (aw) of the powder is also low, notably around 0.1-0.4. In a preferred embodiment, the composition of the invention has therefore a shelf-life of at least 2 years.

In another preferred embodiment, the said composition is a powder. Preferably, in such powder, the mean particle size is 100-120 μm and the bulk density is 0.4-0.6 g/cm³. Particles sizes can be determined using techniques known by the skilled person, for example a Coulter Counter. More preferably, the composition of the invention is in the form of a free-flowing powder. As used herein, the term “free-flowing powder” is well known to the skilled person and includes particulate materials that can be poured (e.g., from one vessel having an opening area of from about 10 cm² to 50 cm² to another vessel of similar dimensions) without substantial clumping of the particles. In detail, the term “free-flowing” is used for a powdered material that is not sticky, and thus has no or hardly any tendency to agglomerate or to adhere to contact surfaces. The so-called angle of repose, θᵣ, is sometimes used as a measure for the flow properties of powders. The angle of repose is the angle that a cone of powder forms between a flat surface when it is poured onto that surface. Typically, for a free-flowing powder, Or is low, e.g. smaller than 60° or smaller than 45°, such as 40° or less. Preferably, in the present invention, Or is of about 35-40°.

In another embodiment, the above-mentioned composition also comprises:
one or more of an emulsifier, preferably two or three emulsifiers,
one or more of an encapsulation agent, preferably three encapsulation agents,
one or more of an antioxidant, preferably three antioxidants,
one or more of a free-flowing agent, preferably one free-flowing agent, and
one or more of a chelating agent, preferably one chelating agent.

As used herein, “an emulsifier” means an agent that stabilizes an emulsion, notably a surface-active agent that promotes the formation and stabilization of an emulsion. Preferably the emulsifier is chosen among propylene glycol fatty acid esters, mono- and diglycerides of fatty acids, propylene glycol alginate (PGA), polyglycerol polyricinoleate (PGPR), polyglycerol esters of fatty acids, sodium caseinate, citric and fatty acid esters of glycerol, sorbitan monolaurate, sorbitan monopalmitate, sorbitan monostearate, sorbitan tristearate, sorbitan monooleate, diacetyl tartaric acid esters of mono- and diglycerides (DATEM), octyl and decyl glycerate, sodium starch octenylsuccinate, sucrose esters of fatty acid, phospholipids, glyceryl mono- and distearate and lecithin. More preferably the emulsifier is chosen among glyceryl mono- and distearate and lecithin.

As used herein, “an encapsulation agent” means an agent that entraps active ingredients within a carrier material in order to envelop, protect and cover the bioactive substance by a physical barrier. Preferably the encapsulation agent is chosen among an agent including protein or its hydrolysate, and starch or its derivatives. More preferably the encapsulation agent is chosen among whey protein concentrate and/or whey protein isolate and/or whey powder. Even more preferably, the three encapsulation agents are chosen among whey protein concentrate, whey protein isolate and demineralized whey powder.

As used herein, “an antioxidant” means a substance that inhibits the oxidation of other molecule. In the present invention, the specific weight ratio of lactose to mannitol confers antioxidative properties to the composition, and the term “antioxidant” has to be understood as a substance different from sugars and sugar alcohols. Preferably the antioxidant is chosen among sodium ascorbate, L-ascorbyl palmitate, and vitamin E, more preferably natural vitamin E.

As used herein “a free-flowing agent” means an agent that prevents packing of particles and acts as a physical barrier when mass is moving, an agent that coat and smooth the edges of bulk powders reducing inter-particle friction, an agent that adsorb excess moisture from the atmosphere before it can be absorbed by the bulk powder. Preferably the free-flowing agent is tricalcium phosphate.

As used herein, “a chelating agent” means a substance whose molecules can form several bonds to a single metal ion. Preferably the chelating agent is chosen among ascorbic acid, citric acid, sodium citrate, phosphates, and citric esters, and more preferably is sodium citrate.

In another embodiment, in the above-mentioned composition, the combined amount of lactose and the mannitol is from 20 to 60% by weight, preferably from 35 to 55% by weight, more preferably from 35 to 40% by weight. In another embodiment, the above-mentioned composition comprises from 15 to 25% by weight of lactose and from 15 to 25% by weight of mannitol.

In a preferred embodiment, the above-mentioned composition comprises:
from 20 to 30% by weight of an edible oil,
from 15 to 25% by weight of lactose,
from 15 to 25% by weight of mannitol,
from 20 to 40% by weight of an encapsulation agent, notably three encapsulation agents,
from 1 to 5% by weight of an emulsifier, notably two or three emulsifiers,
from 1 to 5% by weight of an antioxidant, notably three antioxidants,
from 0.5 to 5% by weight of a free-flowing agent, and
from 1 to 5% by weight of a chelating agent.

In the composition of the invention, the total amount of the components is 100%. In a most preferred embodiment, the above-mentioned composition comprises:
from 20 to 30% by weight of a microalgal oil,
from 15 to 25% by weight of lactose,
from 15 to 25% by weight of mannitol,
from 30 to 40% by weight of whey protein concentrate, whey protein isolate and demineralized whey powder,
from 1 to 5% by weight of glyceryl mono- and distearate and lecithin,
from 1 to 5% by weight of sodium ascorbate, L-ascorbyl palmitate and vitamin E,
from 0.5 to 5% by weight of tricalcium phosphate, and
from 1 to 5% by weight of sodium citrate.

In an even most preferred embodiment, the above-mentioned composition comprises:
25% by weight of a microalgal oil having at least 30% by weight of DHA,
19% by weight of lactose,
19% by weight of mannitol,
12% by weight of whey protein concentrate,
5% by weight of whey protein isolate,
15% by weight of demineralized whey powder,
0.60% by weight of glyceryl mono- and distearate,
0.40% by weight of lecithin,
1% by weight of sodium ascorbate,
0.15% by weight of L-ascorbyl palmitate,
0.06% by weight of vitamin E,
0.50% by weight of tricalcium phosphate, and
2% by weight of sodium citrate.

The compositions of the invention have a good stability to oxidation. Thus, in an embodiment, the oil component of the composition has a peroxide value less or equal to 1 Meq/kg based on ISO 3960 at time 0 (i.e. initial time before storage at an elevated temperature). In another embodiment, the oil component of the composition has a peroxide value less or equal to 5 Meq/kg, notably less or equal to 3 Meq/kg, based on ISO 3960 after 12 days of exposure to air at 65° C. or after 48 days of exposure to air at 45° C. In another embodiment, the oil component of the composition has a p-anisidine value less or equal to 20 Meq/kg, preferably less or equal to 10, more preferably less or equal to 8, and even more preferably less or equal to 7, based on ISO 6885 after 12 days of exposure to air at 65° C. or after 48 days of exposure to air at 45° C. As used herein “ISO 3960” (notably ISO3960:2017) specifies a method for the iodometric determination of the peroxide value of animal and vegetable fats and oils with a visual endpoint detection. The peroxide value is a measure of the amount of oxygen chemically bound to an oil or fat as peroxides, particularly hydroperoxides. As used herein “based on ISO 3960” means “as determined by the method described in ISO 3960”. In a preferred embodiment, when the peroxide value is determined in powder samples, a pre-treatment step can be carried out. This pre-treatment step can be performed as follows. A powder sample (40-50 g, accurately weighed to 0.01 g) is added into 50 mL distilled water inside a 500 mL separatory funnel. The mixture is gently shaken to disperse the powder in the water. Then, 120 mL petroleum ether (having a boiling range of 30° C./60° C.) is added to the mixture and it is shaken to emulsify. After adding 150 mL anhydrous ethanol, the separatory funnel is reversed two times, and is set aside until two phases are completely separated. The upper liquid is taken out and divided into two parts (part A: 20 mL and part B: 40 mL). Part A is dried at 105° C. after the solvent has been evaporated, and then weighed. Part B is put into a 250 mL iodine flask, and then the solvent is evaporated using a rotary evaporator at 50/60° C. Other steps are carried out according to the methods disclosed in ISO 3960 or GB5009.227-2016. As used herein “ISO 6885” (notably ISO 6685:2016) specifies a method for the determination of the p-anisidine value in animal and vegetable fats and oils. This is a measure of the amount of aldehydes present (principally α- and (3-unsaturated aldehydes). As used herein, the term “oil component of the composition” means the oil extracted from the composition by using petroleum ether, because the ISO method is a measure, respectively of the peroxide value or of the p-anisidine value, in oil. Therefore, in order to use the ISO methods, an extraction of the oil from the powder is necessary.

The composition of the invention is also characterized by a surface oil value less or equal to 1% at time 0 (i.e. before the storage) and/or after 12 days of exposure to air at 65° C. or after 48 days of exposure to air at 45° C. As used herein “surface oil” means free oil on the surface of the microencapsulated powder that is not encapsulated in the core materials. It is an indicator to evaluate the efficiency of the microencapsulation: a high amount of free oil indicates poor encapsulation. This surface oil can be easily extracted by using a solvent. More specifically, two extractions with petroleum ether are achieved, then the two solvent phases are combined, evaporated, dried in a vacuum oven. Therefore the free oil ratio of total powder can be calculated.

The composition of the invention is also characterized by a stable DHA content, notably a DHA content comprised between 7 to 8% by weight of the microencapsulated powder. This means that there is no DHA loss in the compositions of the invention (as well as in powder thereof). DHA content can be measured by the following method. Takadiastase is added into the composition (notably in form of a powder), and the mixture is dissolved in distilled water.

The oil in the mixture is extracted three times using ammonia, ethanol, ether, and petroleum ether, subsequently. The residual solvents are then evaporated and vacuum dried to recover the oil for further fatty acid composition analysis. After the oil is methyl esterified and saponified using KOH-methanol followed by dissolution in n-heptane. The DHA content is determined from the fatty acid composition. The method described in the China food safety standard, under reference GB 5009.168-2016, can also be used.

In a second aspect, the present invention also relates to a process of producing the composition of the invention.

Therefore, the present invention relates to a process of producing a composition comprising edible oil, lactose, mannitol, comprising the steps of producing an emulsion or aqueous dispersion of the ingredients contained in the composition; and spray-drying said emulsion or aqueous dispersion. Drying is preferably carried out by spray drying. Conditions for spray drying are known, or can be readily determined by the skilled person. Spray drying is preferably carried out under conditions such that the resulting powder has a mean particle size of 100-120 μm. The mixture may be prepared by combining the components of the composition, such as edible oil, lactose and mannitol, and optionally stirring to form an emulsion. The particulate material (i.e., powder) is thus formed by spray drying of the said emulsion and collected.

In a preferred embodiment, the composition of the invention is obtained by mixing from 10 to 40% by weight of an edible oil, lactose, mannitol, one or more of an encapsulation agent (preferably three encapsulation agents), one or more of an emulsifier (preferably two or three emulsifiers), one or more of an antioxidant (preferably three antioxidants), one or more of a free-flowing agent (preferably one free-flowing agent) and one or more of a chelating agent (preferably one chelating agent).

In a most preferred embodiment, the composition of the invention is obtained by mixing 25% by weight of a microalgal oil having at least 30% by weight of DHA; 19% by weight of lactose; 19% by weight of mannitol; 12% by weight of whey protein concentrate; 5% by weight of whey protein isolate; 15% by weight of demineralized whey powder; 0.60% by weight of glyceryl mono- and distearate; 0.40% by weight of lecithin; 1% by weight of sodium ascorbate; 0.15% by weight of L-ascorbyl palmitate; 0.06% by weight of vitamin E; 0.50% by weight of tricalcium phosphate, and 2% by weight of sodium citrate.

In a third aspect, the present invention also relates to the use of a mixture of lactose and mannitol, in a respective weight ratio from 1:3 to 3:1, preferably between 1:2 and 2:1, more preferably 1:1 to increase the stability against oxidation of a composition comprising from 10 to 40% by weight of an edible oil, said edible oil comprising at least 20% by weight of polyunsaturated fatty acids. The stability against the oxidation can be determined by a sensory evaluation (the composition has good taste and/or odour, even after a storage) and/or by measurement of the peroxide value and/or the p-anisidine value. For example, a composition having a peroxide value less or equal to 1 Meq/kg based on ISO 3960 at time 0, or a peroxide value less or equal to 5 Meq/kg based on ISO 3960 after 12 days of exposure to air at 65° C. or after 48 days of exposure to air at 45° C., and/or ap-anisidine value less or equal to 20, preferably less or equal to 10, more preferably less or equal to 8, even more preferably less or equal to 7, based on ISO 6885 after 12 days of exposure to air at 65° C. or after 48 days of exposure to air at 45° C. are considered to have an increased stability against oxidation.

In a fourth aspect, the present invention also relates to the use of the composition as mentioned above, for the preparation of a nutritional supplement or a dietary product, particularly a nutritional beverage and a chewable tablet. The compositions of the invention can be consumed as is, but they are typically incorporated into a food product or a nutritional supplement before consumption.

In a fifth aspect, the present invention relates to a food or a nutritional product comprising up to 100% by weight of the composition as mentioned above. For example, the present invention relates to a food or a nutritional product comprising at least 5%, 10%, 15%, 20%, 25%, 30%, 35%, 40%, 45%, 50%, 55%, 60%, 65%, 70%, 75%, 80%, 85%, 90%, 95% or 100%, notably at least 50%. The amount of the composition that is present in the food product will depend on the nature of the food product itself. For example, relatively high amounts of the composition may be tolerated in bakery products while smaller amounts are required in certain beverages. Suitable food products include, for example, bakery products (e.g., bread, biscuits or cookies, snack bars), oil-based products (e.g., spreads, salad dressings), dairy products (e.g., milk, reconstitutable milk products, yoghurt, ice cream), infant formulas (which are liquids or reconstituted powders fed to infants and young children) and non-dairy beverages (e.g., fruit juice).

The Figures and following Examples are provided in order to demonstrate and further illustrate certain embodiments and aspects of the present invention and are not to be construed as limiting the scope thereof.

### FIGURES

FIG. 1a represents the results relative to the surface oil change with storage time at 65° C. for the three compositions (control, 50% replacement of lactose, 100% replacement of lactose).

FIG. 1b represents the results relative to the total oxidation change with storage time at 65° C. for the three compositions (control, 50% replacement of lactose, 100% replacement of lactose).

FIG. 1c represents the results relative to the DHA content change with storage time at 65° C. for the three compositions (control, 50% replacement of lactose, 100% replacement of lactose).

FIG. 2 (a to i) represents the texture change with storage time at 65° C. for the three compositions (control, 50% replacement of lactose, 100% replacement of lactose).

### EXAMPLES

### Example 1

### Effect of the Mannitol on DHA Stability by Accelerated Condition

To study the effect of mannitol and its mixture with lactose on the stability of DHA compositions and powder thereof, 0%, 50% and 100% of lactose were replaced with mannitol. Tested compositions are described in the Table 1 below.

TABLE 1
Tested compositions with different ratios of lactose to mannitol

| | Control | 50% | 100% |
|---|---|---|---|
| | (100% | replacement | replacement |
| Ingredients | lactose) | of lactose | of lactose |
| Whey protein concentrate | 12.00% | 12.00% | 12.00% |
| (WPC) | | | |
| Whey protein isolate (WPI) | 5.00% | 5.00% | 5.00% |
| Demineralized whey powder | 15.00% | 15.00% | 15.00% |
| (D90) | | | |
| Sodium ascorbate | 1.00% | 1.00% | 1.00% |
| Sodium Citrate | 2.00% | 2.00% | 2.00% |
| Lecithin | 0.40% | 0.40% | 0.40% |
| Glyceryl mono- and distearate | 0.60% | 0.60% | 0.60% |
| L-ascorbyl palmitate (L-AP) | 0.15% | 0.15% | 0.15% |
| Natural vitamin E | 0.06% | 0.06% | 0.06% |
| Ttricalcium phosphate | 0.50% | 0.50% | 0.50% |
| Edible oil (at least 30% DHA) | 25.16% | 25.16% | 25.16% |
| Lactose | 38.13% | 19.06% | 0.00% |
| Mannitol (Pearlitol 200SD) | 0.00% | 19.06% | 38.13% |
| Total | 100.00% | 100.00% | 100.00% |

1. Process of the Compositions

The compositions, and powder thereof, were obtained by the process described as follows:

All of the water soluble ingredients were dissolved in pure water (to make 45-50% dry substance in the end, for example 100-122 g of water for 100 g of composition) and pasteurized. Edible oil containing DHA were mixed together with other oil soluble ingredients to produce a homogenous solution. The prepared water phase and oil phase were mixed. An emulsion was created by high-speed shearing the mixture until no obvious oil drops (i.e. no oil drops are visible to the naked eye), and then homogenized. The emulsion was spray dried, and tricalcium phosphate was added to the spray-dried powder.

2. Analytical Method

Compositions and powders thereof were then analyzed. Standard values are indicated in Table 2.
Accelerated stability test (exposed to air, 65° C., 12 days)

TABLE 2
Standard values (after accelerated stability test condition)

| | | Analytical |
|---|---|---|
| Items | Unit | method |
| Sensory | / | GB/T5525-2008 |
| | | (panel of 5 persons) |
| Surface oil | w/w % | Extraction by using a |
| | | solvent |
| Peroxide value | Meq/kg | GB5009.227-2016 or |
| | | ISO 3960 |
| p-Anisidine value | / | GB/T24304-2009 or |
| | | ISO 6885 |

3. Results

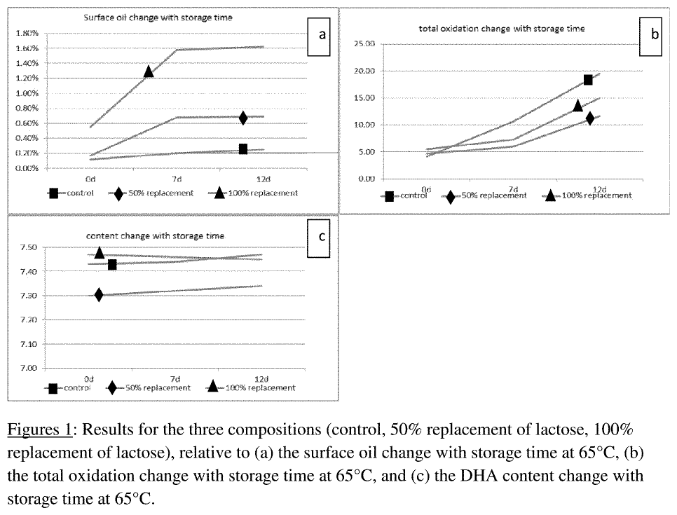

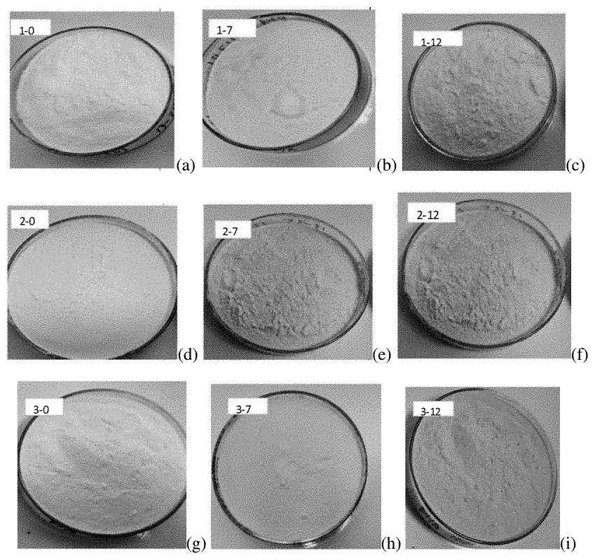

The results are synthetized in Table 3 below, and in FIGS. 1 and 2.

TABLE 3
Results of sensory, surface oil, peroxide value, p-anisidine
value and DHA content after different storage time under accelerated
stability test condition
Control (100% lactose)—accelerated stability test condition (65° C.)

| Storage time | 0 days | 7 days | 12 days |
|---|---|---|---|
| Sensory | White & without | Little fishy & oily | Little fishy & |
| | flavor | odor | oily odor |
| Texture | FIG. 2a | FIG. 2b | FIG. 2c |
| Surface oil | 0.12% | 0.20% | 0.25% |
| Peroxide value | 0.03 | 2.17 | 5.74 |
| p-Anisidine | 4.11 | 6.31 | 8.05 |
| value | | | |
| DHA content | 7.43 | 7.44 | 7.47 |

50% replacement of lactose—accelerated stability
test condition (65° C.)

| | | | |
|---|---|---|---|
| Storage time | 0 days | 7 days | 12 days |
| Sensory | White & without | Little oily & fishy, | Caramel & |
| | flavor | caking | baking odor, |
| | | | caking |
| Texture | FIG. 2d | FIG. 2e | FIG. 2f |
| Surface oil | 0.17% | 0.68% | 0.69% |
| Peroxide value | 0.03 | 0.19 | 2.35 |
| p-Anisidine | 4.63 | 5.64 | 6.91 |
| value | | | |
| DHA content | 7.30 | 7.32 | 7.34 |

100% replacement of lactose—accelerated stability
test condition (65° C.)

| | | | |
|---|---|---|---|
| Storage time | 0 days | 7 days | 12 days |
| Sensory | White & without | Little fishy odor | Caramel & |
| | flavor | | baking odor, |
| | | | little caking |
| Texture | FIG. 2g | FIG. 2h | FIG. 2i |
| Surface oil | 0.55% | 1.58% | 1.62% |
| Peroxide value | 0.03 | 0.73 | 3.58 |
| p-Anisidine | 5.45 | 5.79 | 7.87 |
| value | | | |
| DHA content | 7.47 | 7.46 | 7.45 |

In view of these tests and results, it appears that the three compositions and powder thereof can be classed (from good to bad) as follows:

Sensory:
*Fishy odor: 100% replacement <50% replacement<control
*Caramel color: Control <50% replacement <100% replacement

Surface oil: control <50% replacement <100% replacement

Total oxidation: 50% replacement <100% replacement <control (when assessing the oxidative deterioration of an oil, the total oxidation value or “totox” value (TV) may be helpful.

The calculation is as follows (with the peroxide value expressed in Meq/kg): TV=2*POV+AV; POV being peroxide value and AV being p-anisidine value).

DHA content: no obvious change was found for all three compositions.

### Example 2

### Effect of the Mannitol on DHA Stability at 45° C.

To study the effect of mannitol and its mixture with lactose on the stability of DHA compositions and powder thereof, 0%, 50% and 100% of lactose were replaced with mannitol. Tested compositions are described in the Table 4 below.

TABLE 4
Tested compositions with different ratios of lactose to mannitol

| | Control | 50% | 100% |
|---|---|---|---|
| | (100% | replacement | replacement |
| Ingredients | lactose) | of lactose | of lactose |
| Whey protein concentrate | 12.00% | 12.00% | 12.00% |
| (WPC) | | | |
| Whey protein isolate (WPI) | 5.00% | 5.00% | 5.00% |
| Demineralized whey powder | 15.00% | 15.00% | 15.00% |
| (D90) | | | |
| Sodium ascorbate | 1.00% | 1.00% | 1.00% |
| Sodium Citrate | 2.00% | 2.00% | 2.00% |
| Lecithin | 0.40% | 0.40% | 0.40% |
| Glyceryl mono- and distearate | 0.60% | 0.60% | 0.60% |
| L-ascorbyl palmitate (L-AP) | 0.15% | 0.15% | 0.15% |
| Natural vitamin E | 0.06% | 0.06% | 0.06% |
| Ttricalcium phosphate | 0.50% | 0.50% | 0.50% |
| Edible oil (at least 30% DHA) | 23.85% | 23.85% | 23.85% |
| Lactose | 39.44% | 19.72% | 0.00% |
| Mannitol (Pearlitol 200SD) | 0.00% | 19.72% | 39.44% |
| Total | 100.00% | 100.00% | 100.00% |

1. Process Flowchart of the Compositions

The process is the same as Example 1.

2. Analytical method

Compositions and powders thereof were then analyzed. Standard values are indicated in Table 5.

Main Quality Standard & Analytical Method

TABLE 5
Standard values for stability test at 45° C. for 48 days

| Items | Unit | Analytical method |
|---|---|---|
| Sensory | / | GB/T5525-2008 |
| | | (panel of 5 persons) |
| Surface oil | w/w % | Extraction by using a |
| | | solvent |
| Peroxide value | Meq/kg | GB5009.227-2016 or |
| | | ISO 3960 |
| p-Anisidine value | / | GB/T24304-2009 or |
| | | ISO 6885 |

3. Results

The results are synthetized in Table 6 below.

TABLE 6
Results of sensory, surface oil, peroxide value, and p-anisidine
value after different storage time under stability test
condition at 45° C. for 48 days
Control (100% lactose)—stability test condition at 45° C.

| | | | |
|---|---|---|---|
| Storage time | 0 d | 28 d | 48 d |
| Sensory | characteristic | normal color, slight | normal odor, rancid |
| | DHA odor | fishy odor | oil odor |
| Surface oil | 0.25% | 0.18% | 0.23% |
| Peroxide value | 0.03 | 1.50 | 3.40 |
| p-Anisidine | 4.35 | 6.79 | 7.50 |
| value | | | |

50% replacement of lactose—stability test condition at 45° C.

| | | | |
|---|---|---|---|
| Storage time | 0 d | 28 d | 48 d |
| Sensory | characteristic | slight yellow | slight yellow, little |
| | DHA odor | powder, little fishy | fishy odor, caramel |
| | | and caramel odor | odor |
| Surface oil | 0.24% | 0.25% | 0.34% |
| Peroxide value | 0.03 | 0.84 | 1.40 |
| p-Anisidine | 4.19 | 5.84 | 6.02 |
| value | | | |

100% replacement of lactose—stability test condition at 45° C.

| | | | |
|---|---|---|---|
| Storage time | 0 d | 28 d | 48 d |
| Sensory | characteristic | slight red, little | red, burning odor |
| | DHA odor | caramel odor | |
| Surface oil | 0.41% | 0.69% | 1.34% |
| Peroxide value | 0.03 | 2.57 | 1.45 |
| p-Anisidine | 3.39 | 6.97 | 7.40 |
| value | | | |

In view of these tests and results, it appears that the three compositions and powder thereof can be classed (from good to bad) as follows:

Sensory:
*Fishy odor: 100% replacement <50% replacement <control
*Caramel color: Control <50% replacement <100% replacement

Surface oil: control <50% replacement <100% replacement

Total oxidation: 50% replacement <100% replacement <control (when assessing the oxidative deterioration of an oil, the total oxidation value or “totox” value (TV) may be helpful. The calculation is as follows (with the peroxide value expressed in Meq/kg): TV=2*POV+AV; POV being peroxide value and AV being p-anisidine value).

More precisely, relative to the value of peroxide and p-anisidine, it appears that the composition having 50% replacement of lactose by mannitol led to the better results. This corresponds to a weight ratio of lactose to mannitol of 1:1.

1. A composition comprising:
(i) from 10 to 40% by weight, preferably from 20 to 30% by weight of an edible oil;
(ii) lactose; and
(iii) mannitol,
wherein the edible oil comprises at least 20% by weight, preferably between 25 and 40% by weight, typically 30 and 35% by weight of polyunsaturated fatty acids and the weight ratio of lactose to mannitol is from 1:3 to 3:1, preferably between 1:2 to 2:1, more preferably 1:1.

2. The composition according to claim 1, wherein the edible oil is a fish oil, a krill oil, a microalgal oil, preferably a microalgal oil.

3. The composition according to claim 1, wherein the polyunsaturated fatty acid are chosen in the group consisting of docosahexaenoic acid (DHA), eicosapentaenoic acid (EPA), arachidonic acid (ARA), docosapentaenoic acid (DPA) stearidonic acid, linoleinic acid and a mixture thereof, preferably selected from DHA, EPA and mixtures thereof

4. The composition according to claim 1, wherein the composition is spray-dried.

5. The composition according to claim 1, wherein the composition is a powder.

6. The composition according to claim 1, comprising in addition to (i), (ii) and (iii):
one or more of an emulsifier, such as glyceryl mono- and distearate, lecithin,
one or more of an encapsulation agent, for example an agent including protein or its hydrolysate, such as whey protein, and starch or its derivatives,
one or more of an antioxidant, such as sodium ascorbate, L-ascorbyl palmitate, vitamin E,
one or more of a free-flowing agent, such as tricalcium phosphate, and
one or more of a chelating agent, such as ascorbic acid, citric acid, sodium citrate, phosphates, and citric esters.

7. The composition according to claim 1, wherein the combined amount of lactose and the mannitol is from 20 to 60% by weight, preferably from 35 to 55% by weight, more preferably from 35 to 40% by weight.

8. A process of producing a composition comprising edible oil, lactose, mannitol, comprising the steps of producing an emulsion or aqueous dispersion of the ingredients as defined in claim 1; and spray-drying said emulsion or aqueous dispersion.

9. A method to increase the stability against oxidation of a composition comprising from 10 to 40% by weight of an edible oil, said edible oil comprising at least 20% by weight of polyunsaturated fatty acids, comprising the step of admixing into said composition lactose and mannitol, in a respective weight ration from 1:3 to 3:1 preferably between 1, 2 and 2.1, more preferably 1:1.

10. A nutritional supplement or a dietary product, particularly a nutritional beverage and a chewable tablet, comprising the composition according to claim 1.

11. A food or nutritional product comprising up to 100% by weight of the composition according to claim 1.
